(19) United States
(12) Patent Application Publication
(10) Pub. No.: US 2012/0220554 A1
(43) Pub. Date: Aug. 30, 2012

(54) Combretastatins for Prevention of Posterior Capsule Opacification

(75) Inventors: Roy Quinlan, Durham (GB); Frederique Tholozan, Durham (GB)

(73) Assignee: OXIGENE, INC., South San Francisco, CA (US)

(21) Appl. No.: 13/342,158

(22) Filed: Jan. 2, 2012

(30) Foreign Application Priority Data
Feb. 7, 2010 (US) PCT/US2010/040943

(51) Int. Cl. A61K 31/661; A61P 27/02; C07C 43/215; A61K 31/09; C07F 9/12

(52) U.S. Cl. 514/130; 514/720; 558/197; 568/646

(57) ABSTRACT

Methods and compositions for the treatment and prevention of posterior capsular opacification are provided. The method comprises administering a therapeutically effective amount of a combretastatin to a subject suffering from or at risk of developing posterior capsule opacification.

This application claims the benefit of U.S. provisional patent application No. 61/222,841, filed 2 Jul. 2009, which is incorporated herein by reference in its entirety.

### I. BACKGROUND

Age-related cataract accounts for 50% of blindness worldwide (WHO Fact Sheet N143). The most common complication of cataract surgery is posterior capsular opacification (PCO), which results from the proliferation, migration and epithelial-mesenchymal trans-differentiation of those lens epithelial cells that remain in the eye lens capsular bag after surgery. PCO leads to capsule wrinkling, loss of transparency and the need for further surgery.

Cataract is an extremely prevalent disease—every year there are 250,000 new cases alone in the UK and although there have been advances in the design of intraocular lenses to reduce posterior capsular opacification (PCO), a pharmacological solution to prevent PCO in conjunction with existing technology is desirable. Combretastatin A 4-phosphate (CA4P) inhibits the polymerization of microtubules, and as demonstrated herein, inhibits lens epithelial cell proliferation.

### II. BRIEF DESCRIPTION OF THE DRAWINGS

FIG. 1 illustrates the cell number at 96 h as a percentage of the cell number at 48 h after exposure to paclitaxel or CA4P.

FIG. 2 provides the percentage increase/decrease in cell number for the treated capsules compared to the untreated controls at 48 h after exposure to paclitaxel or CA4P.

FIG. 3 provides photographs of bovine lens cell cultures untreated or treated with 10 μM CA4P at 48 and 96 h after exposure to the test agent.

FIG. 4 provides anti-tubulin western lot of BIE cells 30 minutes, 24 hours and 48 hours after a 30 minute exposure to 1 mM CA4P.

FIG. 5 provides photographs of primary lens epithelial cells cultured on the lens capsule, untreated or treated with 1 mM CA4P for 30 minutes and then grown in culture medium for 48 hours.

FIG. 6 illustrates the cell number over 72 h after a 30 minute exposure to 1 mM CA4P.

FIG. 7 illustrates phosphorylated Erk ½ levels in BIE immediately after being exposed to 1 mM CA4P for 30 min (FIG. 7). GAPDH levels are used for normalization as quantified by densitometry.

### III. SUMMARY OF THE INVENTION

One aspect of the present disclosure provides methods of treating and/or preventing posterior capsular opacification (PCO) comprising administering a therapeutically effective amount of a combretastatin to a subject suffering from or at risk of developing posterior capsule opacification. In certain implementations the combretastatin is combretastatin A-4, or a derivative, prodrug or analog thereof. In a certain implementation, the combretastatin is combretastatin A-4 phosphate (CA4P) or a pharmaceutically acceptable salt thereof. The combretastatin can be administered systemically, e.g. intravenously, or non-systemically, e.g. topically, to an eye of the subject. In certain implementations, the combretastatin is administered during surgery to insert an intraocular lens, optionally followed by subsequent administration of the same or different dose systemically or topically to the eye of the subject.

The present disclosure also provides the use of a combretastatin for the manufacture of a medicament for the treatment or prevention of posterior capsular opacification.

### IV. DETAILED DESCRIPTION

A. Combretastatins

Most generally, any combretastatin or derivative thereof currently known or yet to be discovered that is useful in treating, preventing, or inhibiting posterior capsular opacification may be used in the compositions, liquid formulations, and methods described herein.

Derived from the South African tree Combreturn caffrum, combretastatins such as combretastatin A-4 (CA4), were initially identified in the 1980's as potent inhibitors of tubulin polymerization. CA4 and other combretastatins (e.g. combretastatin A-1 (CA1)) have been shown to bind a site at or near the colchicine binding site on tubulin with high affinity. In vitro studies clearly demonstrated that combretastatins are potent cytotoxic agents against a diverse spectrum of tumor cell types in culture. CA4P and CA1P, respective phosphate prodrugs of CA4 and CA1, were subsequently developed to combat problems with aqueous insolubility (see U.S. Pat. Nos. 4,996,237; 5,409,953; and 5,569,786, each of which is incorporated herein by reference). CA1P and CA4P have also been shown to cause a rapid and acute shutdown of the blood flow to tumor tissue that is separate and distinct from the anti-proliferative effects of the agents on tumor cells themselves. A number of studies have shown that combretastatins cause extensive shut-down of blood flow within tumor microvasculature, leading to secondary tumor cell death (Dark et al., Cancer Res. 57: 1829-34, (1997); Chaplin et al., Anticancer Res. 19: 189-96, (1999); Hill et al., Anticancer Res. 22(3):1453-8 (2002); Holwell et al., Anticancer Res. 22(2A):707-11, (2002). Blood flow to normal tissues is generally far less affected by CA4P and CA1P than blood flow to tumors (Tozer et al., Cancer Res. 59: 1626-34 (1999)).

CA4P and CA4 disrupt microtubule assembly leading to the collapse of the nascent endothelial cell cytoskeleton (mature endothelial cell shape is maintained by the secondary scaffolding protein actin). These newly formed or abnormal endothelial cells then change shape from flat and elongated to rounded or spherical. This endothelial cell shape alteration causes vascular occlusion in immature and abnormal blood vessels, but has no effect on normal mature blood vessels. Selectivity depends on the differentiated state as much as on the age of the endothelial call. For example, in tumor vasculature, mature endothelial cells are structurally abnormal and lack an actin cytoskeleton, rending them sensitive to CA4P. Without being bound to a theory of the invention, it is believed that this selectivity for immature epithelial cells cause CA4P and other combretastatins to be particularly effective in preventing PCO.

In particular embodiments, the combretastatin is a compound of Formula I:

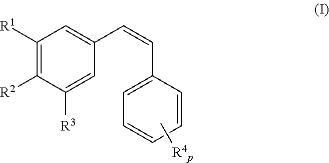

wherein
each of R¹, R² and R³, independently of the others, is selected from the group consisting of hydrogen, C₁₋₆ alkoxy, and halogen, wherein at least two of R¹, R² and R³ are non-hydrogen;
R⁴ is selected from the group consisting of R⁵, R⁶, R⁵ substituted with one or more of the same or different R⁷ or R⁶, —OR′ substituted with one or more of the same or R⁷ or R⁶, —B(OR⁷)₂, —B(NR⁸R⁸)₂, —(CH₂)ₘ—R⁶, —(CHR⁷)ₘ—R⁶, —O—(CH₂)ₘ—R⁶, —S—(CH₂)ₘ—R⁶, —O—CHR⁷R⁶, —O—CR⁷(R⁶)₂, —O—(CHR⁷)ₘ—R⁶, —O— (CH₂)ₘ—CH[(CH₂)ₘR⁶]R⁶, —S—(CHR⁷)ₘ—R⁶, —C(O)NH—(CH₂)ₘ—R⁶, —C(O)NH—(CHR⁷)ₘ—R⁶, —O—(CH₂)ₘ—C(O)NH—(CH₂)ₘ—R⁶, —S—(CH₂)ₘ—C(O)NH—(CH₂)ₘ—R⁶, —O—(CHR⁷)ₘ—C(O)NH—(CHR⁷)ₘ—R⁶, —S—(CHR⁷)ₘ—C(O)NH—(CHR⁷)ₘ—R⁶, —NH—(CH₂)ₘ—R⁶, —NH—(CHR⁷)ₘ—R⁶, —NH[(CH₂)ₘR⁶], —N[(CH₂)ₘR⁶]₂, —NH—C(O)—NH—(CH₂)ₘ—R⁶, —NH—C(O)—(CH₂)ₘ—CHR⁶R⁶ and —NH—(CH₂)ₘ—C(O)—NH—(CH₂)ₘ—R⁶;
each R⁵ is independently selected from the group consisting of C₁₋₆ alkyl, C₃₋₈ cycloalkyl, C₄₋₁₁ cycloalkylalkyl, C₅₋₁₀ aryl, C₆₋₁₆ arylalkyl, 2-6 membered heteroalkyl, 3-8 membered heterocyclyl, 4-11 membered cycloheteroalkylalkyl, 5-10 membered heteroaryl, 6-16 membered heteroarylalkyl, phosphate, phosphate ester, phosphonate, phosphorodiamidate, phosphoramidate monoester, phosphoramidate diester, cyclic phosphoramidate, cyclic phosphorodiamidate, and phosphonamidate each R⁶ is a suitable group independently selected from the group consisting of ═O, —OR′, C₁₋₃ haloalkyloxy, —OCF₃, ═S, —SR′, ═NR⁷, ═NOR′, —NR⁸R⁸, halogen, —CF₃, —CN, —NC, —OCN, —SCN, —NO, —NO₂, ═N₂, —N₃, —S(O)R⁷, —S(O)₂R⁷, —S(O)₂OR⁷, —S(O)NR⁸R⁸, —S(O)₂NR⁸R⁸, —OS(O)R⁷, —OS(O)₂R⁷, —OS(O)₂OR⁷, —OS(O)₂NR⁸R⁸, —C(O)R⁷, —C(O)OR⁷, —C(O)NR⁸R⁸, —C(NH)NR⁸R⁸, —C(NR⁷)NR⁸R⁸, —C(NOH)R⁷, —C(NOH)NR⁸R⁸, —OC(O)R⁷, —OC(O)OR⁷, —OC(O)NR⁸R⁸, —OC(NH)NR⁸R⁸, —OC(NR⁷)NR⁸R⁸, —[NHC(O)]ₙR⁷, —[NR⁷C(O)]ₙR⁷, —[NH C(O)]ₙOR⁷, —[NR⁷C(O)]ₙOR⁷, —[NHC(O)]ₙNR⁸R⁸, —[NR⁷C(O)]ᵣ/NR⁸R⁸, —[NHC(NH)]ₙNR⁸R⁸ and —[NR⁷C(NR⁷)]ₙNR⁸R⁸;
each R⁷ is independently selected from the group consisting of hydrogen, C₁₋₆ alkyl, C₃₋₈ cycloalkyl, C₄₋₁₁ cycloalkylalkyl, C₅₋₁₀ aryl, C₆₋₁₆ arylalkyl, 2-6 membered heteroalkyl, 3-8 membered cycloheteroalkyl, 4-11 membered cycloheteroalkylalkyl, 5-10 membered heteroaryl, 6-16 membered heteroarylalkyl, phosphate, phosphate ester, phosphonate, phosphorodiamidate, phosphoramidate monoester, phosphoramidate diester, cyclic phosphoramidate, cyclic phosphorodiamidate, and phosphonamidate;
each R⁸ is independently R⁷ or, alternatively, two R⁸ are taken together with the nitrogen atom to which they are bonded to form a 5 to 8-membered heterocyclyl or heteroaryl which may optionally include one or more of the same or different additional heteroatoms and which may optionally be substituted with one or more of the same or different R⁷ or suitable R⁶ groups;
each m independently is an integer from 1 to 3;
each n independently is an integer from 0 to 3;
p is an integer from 1 to 5, and
   wherein two adjacent R⁴ groups and their intervening atoms can be bonded to form a 5-8 membered ring fused to the central phenyl group.

In a particular embodiment, the combretastatin derivative is a phosphate prodrug of a combretastatin. An exemplary phosphate prodrug is a compound of the Formula II:

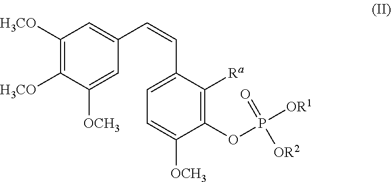

wherein
R^{a} is H or OP(O)(OR³)OR⁴; and
OR¹, OR², OR³ and OR⁴ are each, independently, OH, —O⁻QH⁺ or —O⁻M⁺, wherein M⁺ is a monovalent or divalent metal cation and Q is, independently:
   a) an amino acid containing at least two nitrogen atoms where one of the nitrogen atoms, together with a proton, forms a quaternary ammonium cation QH⁺; or
   b) an organic amine containing at least one nitrogen atom which, together with a proton, forms a quaternary ammonium cation, QH⁺.
   In one embodiment of Formula II, R^{a} is H, one of OR¹ and OR² is hydroxyl, and the other is —O⁻QH⁺ where Q is tris(hydroxymethyl)amino methane (TRIS″ or “tromethamine”).

In another embodiment of Formula II, R^{a} is H or OP(O)(OR³)OR⁴, and R¹, R², R³ and R⁴ are each, independently, an aliphatic organic amine, alkali metals, transition metals, heteroarylene, heterocyclyl, nucleoside, nucleotide, alkaloid, amino sugar, amino nitrile, or nitrogenous antibiotic.

In another embodiment of Formula II, R¹, R², R³ and R⁴ are each, independently, Na, tromethamine, histidine, ethanolamine, diethanolamine, ethylenediamine, diethylamine, triethanolamine, glucamine, N-methylglucamine, ethylenediamine, 2-(4-imidazolyl)-ethylamine, choline, or hydrabamine.

In another embodiment, Formula II is represented by a compound of Formula III:

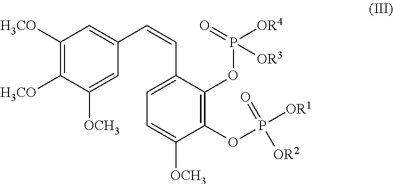

wherein
OR¹, OR², OR³ and OR⁴ are each, independently, OH, —O⁻QH⁺ or —O⁻M⁺, wherein M⁺ is a monovalent or divalent metal cation, and Q is, independently:
   a) an amino acid containing at least two nitrogen atoms where one of the nitrogen atoms, together with a proton, forms a quaternary ammonium cation QH⁺; or
   b) an organic containing at least one nitrogen atom which, together with a proton, forms a quaternary ammonium cation, QH⁺.

In one embodiment of Formula III, at least one of OR¹, OR², OR³ and OR⁴ is hydroxyl, and at least one of OR¹, OR², OR³ and OR⁴ is —O⁻QH⁺, where Q is tromethamine.

In one exemplary embodiment, a combretastatin derivative is the amine or serinamide derivative of CA4, e.g. AVE8032 (Aventis Pharma, France). In another exemplary embodiment, a combretastatin derivative is ZD6126 (AstraZeneca, UK).

Exemplary combretastatin salts contemplated for use in the methods described herein are discussed in detail in WO 99/35150; WO 01/81355; U.S. Pat. Nos. 6,670,344; 6,538,038; 5,569,786; 5,561,122; 5,409,953; 4,996,237 which are incorporated herein by reference in their entirety.

Other combretastatin derivatives or analogs of combretastatins that can be used in the methods disclosed herein are described in Singh et al., J. Org. Chem., 1989; Cushman et al, J. Med. Chem., 1991; Getahun et al, J. Med. Chem., 1992; Andres et al, Bioorg. Med. Chem. Lett., 1993; Mannila, et al., Liebigs. Ann. Chem., 1993; Shirai et al., Bioorg. Med. Chem. Lett., 1994; Medarde et al., Bioorg. Med. Chem. Lett., 1995; Wood et al, Br. J. Cancer, 1995; Bedford et al., Bioorg. Med. Chem. Lett., 1996; Dorr et al., Invest. New Drugs, 1996; Jonnalagadda et al., Bioorg. Med. Chem. Lett., 1996; Shirai et al., Heterocycles, 1997; Aleksandrzak, et al., Anticancer Drugs, 1998; Chen et al., Biochem. Pharmacol., 1998; Ducki et al., Bioorg. Med. Chem. Lett., 1998; Hatanaka et al., Bioorg. Med. Chem. Lett., 1998; Medarde et al., Eur. J. Med. Chem., 1998; Medina et al., Bioorg. Med. Chem. Lett., 1998; Ohsumi et al., Bioorg. Med. Chem. Lett., 1998; Ohsumi et al., J. Med. Chem., 1998; Pettit, et al., J. Med. Chem., 1998; Shirai et al., Bioorg. Med. Chem. Lett., 1998; Banwell et al., Aust. J. Chem., 1999; Medarde et al., Bioorg. Med. Chem. Lett., 1999; Shan et al., PNAS, 1999; Combeau et al., Mol. Pharmacol., 2000; Pettit et al., J. Med. Chem., 2000; Pinney et al., Bioorg. Med. Chem. Lett., 2000; Flynn et al., Bioorg. Med. Chem. Lett., 2001; Gwaltney et al., Bioorg. Med. Chem. Lett., 2001; Lawrence et al., 2001; Nguyen-Hai et al., Bioorg. Med. Chem. Lett., 2001; Xia et al., J. Med. Chem., 2001; Tahir et al., Cancer Res., 2001; Wu-Wong et al., Cancer Res., 2001; Janik et al, Biooorg. Med. Chem. Lett., 2002; Kim et al., Bioorg Med Chem. Lett., 2002; Li et al., Biooorg. Med. Chem. Lett., 2002; Nam et al., Bioorg. Med. Chem. Lett., 2002; Wang et al., J. Med. Chem. 2002; Hsieh et al., Biooorg. Med. Chem. Lett., 2003; Hadimani et al., Bioorg. Med. Chem. Lett., 2003; Mu et al., J. Med. Chem, 2003; Nam et al., Curr. Med. Chem., 2003; Pettit et al, J. Med. Chem., 2003; Gaukroger et al., Org Biomol Chem. 2003; Bailly et al., J Med. Chem. 2003; Sun et al., Anticancer Res. 2004; Sun et al., Bioorg Med Chem. Lett. 2004; Liou et al., J Med. Chem. 2004; Perez-Melero et al., Bioorg Med Chem. Lett. 2004; Liou et al., J Med. Chem. 2004; Mamane et al., Chemistry. 2004; De Martini et al, J Med. Chem. 2004; Ducki et al, J Med Chem. 2005; Maya et al., J Med. Chem. 2005; Medarde et al., J Enzyme Inhib Med. Chem. 2004; Simoni et al, J Med. Chem. 2005; Sanchez et al., Bioorg Med. Chem. 2005; Vongvanich et al., Planta Med. 2005; Tron et al., J Med. Chem. 2005; Borrel et al., Bioorg Med. Chem. 2005; Hsieh et al., Curr Pharm Des. 2005; Lawrence et al, Curr Pharm Des. 2005; Hadfield et al., Eur J Med. Chem. 2005; Pettit et al., J Med. Chem. 2005; Coggioloa et al., Bioorg Med Chem. Lett. 2005; Kaffy et al., Org Biomol Chem. 2005; Mateo et al, J Org. Chem. 2005; LeBlanc et al., Bioorg Med. Chem. 2005; Srivistava et al., Bioorg Med. Chem. 2005; Nguyen et al., J Med. Chem. 2005; Kong et al., Chem. Biol. 2005; Li et al, Bioorg Med Chem. Lett. 2005; Pettit et al, J Nat. Prod. 2005; Nicholson et al, Anticancer Drugs. 2006; Monk et al., Bioorg Med. Chem. 2006; De Martino et al., J Med. Chem. 2006; Peifer et al., J Med. Chem. 2006; Kaffy et al., Bioorg Med. Chem. 2006; Banwell et al., Bioorg Med. Chem. 2006; Dupeyre et al., Bioorg Med. Chem. 2006 Simoni et al, J Med. Chem. 2006; Tron et al., J Med. Chem. 2006; Romagnoli et al, J Med. Chem. 2006; Pandit et al., Bioorg Med. Chem. 2006; Nakamura et al., ChemMedChem. 2006; Pirali et al., J Med. Chem. 2006; Bellina et al., Bioorg Med Chem. Lett. 2006; Hu et al, J Med. Chem. 2006; Chang et al., J Med. Chem. 2006; Thomson et al., Mol Cancer Ther. 2006; Fortin et al., Bioorg Med Chem. Lett., 2007; Duan et al., J Med. Chem., 2007; Zhang et al., J Med. Chem. 2007; Wu et al., Bioorg Med Chem. Lett. 2007; Sun et al., Bioorg Med Chem. Lett. 2007, WO 07/140,662; WO 07/059,118; WO 06/138427; WO 06/036743; WO 05/007635, WO 03/040077, WO 03/035008, WO 02/50007, WO 02/14329; WO 01/12579, WO 01/09103, WO 01/81288, WO 01/84929, WO 00/48590, WO 00/73264, WO 00/06556, WO 00/35865, WO 99/34788, WO 99/48495, WO 92/16486, U.S. Pat. Nos. 7,312,241; 7,223,747; 7,220,784; 7,135,502; 7,125,906; 7,105,695; 7,105,501; 7,087,627; 7,030,123; 7,078,552; 7,030,123; 7,018,987; 6,992,106; 6,919,324; 6,846,192, 6,855,702; 6,849,656; 6,794,384; 6,787,672, 6,777,578, 6,723,858, 6,720,323, 6,433,012, 6,423,753, 6,201,001, 6,150,407, 6,169,104, 5,731,353, 5,674,906, 5,430,062, 5,525,632, 4,996,237 and 4,940,726, each of which are incorporated herein by reference in their entirety.

The composition can include additional active agents. Exemplary active agents include analgesics, anesthetics, or anti-inflammatory agents. In some embodiments, active agents that may be used in the liquid formulations are anti-inflammatory agents (such as hydrocortisone, dexamethasone, fluocinolone, prednisone, prednisolone, methylprednisolone, fluorometholone, betamethasone and triamcinolone), ace-inhibitors, endogenous cytokines, agents that influence basement membrane, agents that influence the growth of endothelial cells, adrenergic agonists or blockers, cholinergic agonists or blockers, aldose reductase inhibitors, antibiotics (such as tetracycline, chlortetracycline, bacitracin, neomycin, polymyxin, gramicidin, cephalexin, oxytetracycline, chloramphenicol, rifampicin, ciprofloxacin, aminosides, gentamycin, erythromycin, penicillin, quinolone, ceftazidime, vancomycin, imipeneme, sulfonamides, sulfadiazine, sulfacetamide, sulfamethizole, sulfisoxazole, nitrofurazone and sodium propionate), antifungals (such as amphotericin B, fluconazole, ketoconazole and miconazole), anti-allergics (such as sodium cromoglycate, antazoline, methapyriline, chlorpheniramine, cetirizine, pyrilamine and prophenpyridamine), antihypertensives, pressors, antiprotozoal agents, antiviral agents, antifungal agents, anti-infective agents, antitumor agents, antimetabolites, and antiangiogenic agents.

In some embodiments the combretastatin, used in combination with an additional active agent, may be administered simultaneously (i.e., simultaneous administration), sequentially (i.e., sequential administration), or independently of the additional active agents. In some embodiments the combretastatin and the additional active agent are administered by the same route of administration. In some embodiments, the combretastatin and additional active agent are both administered during one visit to the medical professional. In some embodiments, the combretastatin and additional active agent are both administered during the same physician visit. In some embodiments, the combretastatin and additional active agent are administered during more than one visit to the physician.

In some embodiments the combretastatin and the additional active agent are administered by the same route of administration. In some embodiments the combretastatin and the additional active agent are administered by different routes of administration.

In some embodiments the combination of administration of combretastatin and administration of additional active agent reduces the dose administered (e.g., dose volume, dose concentration, or amount of combretastatin) of one or both agents, as compared to the amount effect when used as a monotherapy. In some embodiments the combination of combretastatin and additional active agent increases or prolongs the time between administrations and/or decreases the frequency of administrations of one or both agents.

In some embodiments, the formulations and pharmaceutical formulations described herein are used to prevent PCO where the subject, including but not limited to a human subject, is at heightened risk of developing PCO. A subject with a heightened risk of developing PCO is a subject with one or more indications that PCO is likely to develop in the particular subject. In some embodiments, a formulation or pharmaceutical formulation comprises a combretastatin compound such as CA4P, and is administered to treat or prevent PCO.

The formulation, liquid formulations and methods described herein can be used to treat, prevent, or inhibit PCO in a subject. Posterior capsular opacification (PCO) is a disorder characterized by hyperplasia and migration of posterior capsular cells, showing up as a thickening, opacification and clouding of the posterior lens capsule, which is left behind when the cataract was removed, for placement of the intraocular lens (IOL). This may compromise visual acuity. Currently there is no method or drug for preventing development of PCO. The current standard treatment of PCO is use of an Nd-YAG laser (neodymium-yttrium-aluminum-garnet) to disrupt and clear the central portion of the opacified posterior lens capsule. In very thick opacified posterior capsules, a manual surgical procedure may be necessary to remove the opacification.

As used herein, to “treat” PCO by administration of a combretastatin means that the progress of at least one detectable physical characteristic or symptom of PCO is slowed, stopped, or reversed following administration of the combretastatin. Exemplary detectable physical characteristics or symptoms of PCO include, without limitation, hyperplasia of posterior capsular cells and loss of visual acuity.

As used herein, to “inhibit” PCO by administration of a combretastatin means that the progress of at least one detectable physical characteristic or symptom of PCO is slowed or stopped following administration of the combretastatin.

As used herein, to “prevent” PCO by administration of a combretastatin means that detectable physical characteristics or symptoms of PCO do not develop following administration of the combretastatin.

A subject, including but not limited to a human subject, having a predisposition or in need of prevention may be identified by the skilled practitioner by established methods and criteria in the field given the teachings herein. The skilled practitioner may also readily diagnose individuals as in need of inhibition or treatment based upon established criteria in the field for identifying PCO or a predisposition to PCO given the teachings herein.

As used herein, a “subject” is generally any animal that may benefit from administration of the combretastatins described herein. In some embodiments the combretastatins are administered to a mammalian subject. In some embodiments the combretastatins are administered to a human subject.

B. Administration

The compositions, methods, and liquid formulations described herein deliver one or more combretastatins to a subject, including but not limited to a human subject.

In some embodiments, the compositions, methods, and liquid formulations described herein deliver one or more combretastatins to an aqueous medium of a human subject.

In some embodiments, the compositions, methods, and liquid formulations described herein deliver one or more combretastatins to an eye of a subject, including directly to the lens capsule, in an amount and for a duration effective to treat, prevent, or inhibit PCO.

As a non-limiting example, the compositions, liquid formulations, and methods described in herein may be administered to the vitreous, aqueous humor, sclera, conjunctiva, between the sclera and conjunctiva, the retina choroid tissues, macula, or other area in or proximate to the eye of a subject, either by direct administration to these tissues or by periocular routes, in amounts and for a duration effective to treat, prevent, or inhibit PCO. The effective amounts and durations may be different for each of treating, preventing, or inhibiting PCO, and for each of the different sites of delivery.

Intravitreal administration is more invasive than some other types of ocular procedures. Because of the potential risks of adverse effects, intravitreal administration may not be optimal for treatment of relatively healthy eyes. By contrast, periocular administration, such as subconjunctival administration, is much less invasive than intravitreal administration. When a combretastatin is delivered by a periocular route, it may be possible to treat patients with healthier eyes than could be treated using intravitreal administration. In some embodiments, subconjunctival injection is used to prevent or delay onset of a disease or condition of the eye, where the eye of the subject has visual acuity of 20/40 or better.

“Subconjunctival” placement or injection, as used herein, refers to placement or injection between the sclera and conjunctiva. Subconjunctival is sometimes referred to herein as “sub-conj” administration.

In some embodiments the combretastatin can be administered prior to, concurrent with, or subsequent to surgical implantation of an intraocular lens. In some embodiments the combretastatin is administered during surgery. In a related embodiment, the combretastatin is administered locally during surgery followed by one or more subsequent doses of the same or different amount, either systemically or topically to the eye. In certain embodiments, the intraocular lens is bathed in combretastatin prior to insertion. The combretastatin can be administered once or in multiple doses.

Routes of administration that may be used to administer a liquid formulation include but are not limited to placement of the liquid formulation, for example by injection, into an aqueous medium in the subject, including but not limited to placement, including but not limited to by injection, into the eye of a subject, including but not limited to a human subject. The liquid formulation may be administered systemically, including but not limited to the following delivery routes: rectal, vaginal, infusion, intramuscular, intraperitoneal, intraarterial, intrathecal, intrabronchial, intracisternal, cutaneous, subcutaneous, intradermal, transdermal, intravenous, intracervical, intraabdominal, intracranial, intraocular, intrapulmonary, intrathoracic, intratracheal, nasal, buccal, sublingual, oral, parenteral, or nebulised or aerosolized using aerosol propellants.

Compositions and liquid formulations comprising combretastatin can be administered directly to the eye using a variety of procedures, including but not limited to procedures in which (1) the combretastatin is administered by injection using a syringe and hypodermic needle, (2) a specially designed device is used to inject the combretastatin, (3) prior to injection of the combretastatin, a pocket is surgically formed within the sclera to serve as a receptacle for the combretastatin or combretastatin composition. For example, in one administration procedure a surgeon forms a pocket within the sclera of the eye followed by injection of a solution or liquid formulation comprising the combretastatin into the pocket.

Other administration procedures include, but are not limited to procedures in which (1) a formulation of the combretastatin is injected through a specially designed curved cannula to place the combretastatin directly against a portion of the eye, (2) a compressed form of the combretastatin is placed directly against a portion of the eye, (3) the combretastatin is inserted into the sclera by a specially designed injector or inserter, (4) the liquid formulation comprising the combretastatin is incorporated within a polymer, (5) a surgeon makes a small conjunctival incision through which to pass a suture and any combretastatin delivery structure so as to secure the structure adjacent to the sclera, (6) a needle is used for injection directly into the vitreous of an eye, or into any other site described.

The liquid formulations described herein may be used directly, for example, by injection, as an elixir, for topical administration including but not limited to via eye drops, or in minitablets.

In some embodiments, the liquid formulations described herein may be administered by topical administration. In some embodiments, the topical administration is ocular topical administration. In some embodiments, the ocular topical administration includes, but is not limited to, administration via eye drops, contacts, punctual plugs, or other ocular devices. In some embodiments, the liquid formulation is applied topically, including topically to the eye, any of about 1, 2, 3, 4, or 5 times per day. In some embodiments, the liquid formulation is applied topically, including topically to the eye, about once or less any of about every 1, 2, 3, 4, 5, 6, 7, 10, 14, 21, or 28 day(s). In some embodiments, the liquid formulation is applied topically, including topically to the eye, about once or less a day. In some embodiments, the liquid formulation is applied topically, including topically to the eye, about once or less every 5 days. In some embodiments, the liquid formulation is applied topically, including topically to the eye, about once or less of every 10 days.

In certain implementations, the combretastatin preferably is administered parenterally, e.g., intravenously, intramuscularly, intravenously, subcutaneously, or intraperitoneally. The carrier or excipient or excipient mixture can be a solvent or a dispersive medium containing, for example, various polar or non-polar solvents, suitable mixtures thereof, or oils. As used herein “carrier” or “excipient” means a pharmaceutically acceptable carrier or excipient and includes any and all solvents, dispersive agents or media, coating(s), antimicrobial agents, iso/hypo/hypertonic agents, absorption-modifying agents, and the like. The use of such substances and the agents for pharmaceutically active substances is well known in the art. Except insofar as any conventional media or agent is incompatible with the active ingredient, use in therapeutic compositions is contemplated. Moreover, other or supplementary active ingredients can also be incorporated into the final composition.

Solutions of the combretastatin may be prepared in suitable diluents such as water, ethanol, glycerol, liquid polyethylene glycol(s), various oils, and/or mixtures thereof, and others known to those skilled in the art.

The pharmaceutical forms suitable for injectable use include sterile solutions, dispersions, emulsions, and sterile powders. The final form must be stable under conditions of manufacture and storage. Furthermore, the final pharmaceutical form must be protected against contamination and must, therefore, be able to inhibit the growth of microorganisms such as bacteria or fungi. A single intravenous or intraperitoneal dose can be administered.

Alternatively, a slow long term infusion or multiple short term daily infusions may be utilized, typically lasting from 1 to 8 days. Alternate day or dosing once every several days may also be utilized.

Sterile, injectable solutions are prepared by incorporating a compound in the required amount into one or more appropriate solvents to which other ingredients, known to those skilled in the art, may be added as required. Sterile injectable solutions are prepared by incorporating the combretastatin in the required amount in the appropriate solvent with various other ingredients as required. Sterilizing procedures, such as filtration, then follow. Typically, dispersions are made by incorporating the compound into a sterile vehicle which also contains the dispersion medium and the required other ingredients as indicated above.

In the case of a sterile powder, the preferred methods include vacuum drying or freeze drying to which any required ingredients are added.

In all cases the final form, as noted, must be sterile and must also be able to pass readily through an injection device such as a hollow needle. The proper viscosity may be achieved and maintained by the proper choice of solvents or excipients. Moreover, the use of molecular or particulate coatings such as lecithin, the proper selection of particle size in dispersions, or the use of materials with surfactant properties may be utilized.

Prevention or inhibition of growth of microorganisms may be achieved through the addition of one or more antimicrobial agents such as chlorobutanol, ascorbic acid, parabens, thimerosal, or the like. It may also be preferable to include agents that alter the tonicity such as sugars or salts.

With mammals, including humans, the effective amounts can be determined by standard method and administered on the basis of body surface area. The interrelationship of dosages varies for animals of various sizes and species, and for humans (based on mg/m² of body surface) is described by E. J. Freireich et al., Cancer Chemother. Rep., 50(4):219 (1966). Body surface area may be approximately determined from the height and weight of an individual (see, e.g., Scientific Tables, Geigy Pharmaceuticals, Ardsley, N.Y. pp. 537-538 (1970)). A suitable dose range is from 1 to 1000 mg of equivalent per m² body surface area of a combretastatin, for instance from 50 to 500 mg/m².

C. Compositions and Formulations

The formulations described herein contain a combretastatin and may generally be any liquid or semi-liquid formulation, including but not limited to solutions, suspensions, emulsions, and gels.

In some embodiments, a total volume of a liquid formulation described herein is topically administered to a subject's eye, including but not limited to a human subject's eye that is less than about 1000 μl, less than about 900 μl, less than about 800 μl, less than about 700 μl, less than about 600 μl, less than about 500 μl, less than about 400 μl, less than about 300 μl, less than about 200 μl, less than about 150 μl, less than about 100 μl, less than about 90 μl, less than about 80 μl, less than about 70 μl, less than about 60 μl, less than about 50 μl, less than about 40 μl, less than about 30 μl, less than about 20 μl, less than about 10 μl, less than about 5 μl, less than about 3 μl, or less than about 1 μl. In some embodiments, a volume of a liquid formulation described herein is topically administered to a subject's eye, including but not limited to a human subject's eye that is less than about 200 μl. In some embodiments, a volume of a liquid formulation described herein is topically administered to a subject's eye, including but not limited to a human subject's eye that is less than about 100 μl. In some embodiments, a volume of a liquid formulation described herein is topically administered to a subject's eye, including but not limited to a human subject's eye that is between about 0.1 μl and about 200 μl, between about 50 μl and about 200 μl, between about 200 μl and about 300 μl, between about 300 μl and about 400 μl, between about 400 μl and about 500 μl, between about 600 μl and about 700 μl, between about 700 μl and about 800 μl, between about 800 μl and about 900 μl, between about 900 μl and about 1000 μl, between about 50 μl and about 150 μl, between about 0.1 μl and about 100 μl, between about 0.1 μl and about 50 μl, between about 1 μl and about 40 μl, between about 1 μl and about 30 μl, between about 1 μl and about 20 μl, between about 1 μl and about 10 μl, or between about 1 μl and about 5 μl. In some embodiments, a total volume of a liquid formulation described herein is topically administered to a subject's eye, including but not limited to a human subject's eye that is between about 10 μl and about 200 μl. In some embodiments, a total volume of a liquid formulation described herein is administered topically to a subject's eye, including but not limited to a human subject's eye that is between about 0.1 μl and about 200 μl. In some embodiments, a total volume of a liquid formulation described herein is topically administered to a subject's eye, including but not limited to a human subject's eye that is between about 40 μl and about 160 μl. In some embodiments, a total volume of a liquid formulation described herein is topically administered to a subject's eye, including but not limited to a human subject's eye that is about 40 μl. In some embodiments, a total volume of a liquid formulation described herein is topically administered to a subject's eye, including but not limited to a human subject's eye that is about 80 μl.

In some embodiments, a total amount of combretastatin less than about 5 mg is administered topically. In some embodiments, a total amount of combretastatin less than about 5.0 mg is administered topically. In some embodiments, a total amount of combretastatin less than about 4.5 mg is administered topically. In some embodiments, a total amount of combretastatin less than about 4.0 mg is administered topically. In some embodiments, a total amount of combretastatin less than about 3.5 mg is administered topically. In some embodiments, a total amount of combretastatin less than about 3.0 mg is administered topically. In some embodiments, a total amount of combretastatin less than about 2.5 mg is administered topically. In some embodiments, a total amount of combretastatin less than about 2 mg is administered topically. In some embodiments, a total amount of combretastatin less than about 1.2 mg is administered topically. In some embodiments, a total amount of combretastatin less than about 1.0 mg is administered topically. In some embodiments, a total amount of combretastatin less than about 0.8 mg is administered topically. In some embodiments, a total amount of combretastatin less than about 0.6 mg is administered topically. In some embodiments, a total amount of combretastatin less than about 0.4 mg is administered topically. In some embodiments, a volume of a formulation is administered that contains an amount of combretastatin described herein. In some embodiments, a total amount of combretastatin administered topically is any of between about 20 μg and about 4000 μg, between about 10 μg and about 2000 μg, between about 10 μg and 1750 μmg, between about 1500 μg and 1000 μg, or between about 10 μg and 1000 μg. In some embodiments, a total amount of combretastatin administered topically is about 1660 μg. In some embodiments, a total amount of combretastatin administered topically is about 880 μg. In some embodiments, a total amount of combretastatin administered topically is 40 μg. In some embodiments, a total amount of combretastatin administered topically is about 28 μg.

When a certain volume of a liquid formulation is administered, it is understood that there is some imprecision in the accuracy of various devices that may be used to administer the liquid formulation. Where a certain volume is specified, it is understood that this is the target volume. However, certain devices such as insulin syringes are inaccurate to greater than 10%, and sometimes inaccurate to greater than 20% or more. Hamilton HPLC type syringes are generally considered precise to within 10%, and are recommended for volumes below 10 μl are to be injected.

In some embodiments, a volume of a liquid formulation described herein is administered to the vitreous of a subject's eye, including but not limiting a human subject's eye that is less than about 500 μl, less than about 400 μl, less than about 300 μl, less than about 200 μl, less than about 100 μl, less than about 90 μl, less than about 80 μl, less than about 70 μl, less than about 60 μl, less than about 50 μl, less than about 40 μl, less than about 30 μl, less than about 20 μl, less than about 10 μl, less than about 5 μl, less than about 3 μl, or less than about 1 μl. In some embodiments, a volume of a liquid formulation described herein is administered to the vitreous of a rabbit eye or subject's, including but not limited to a human subject's eye that is less than about 20 μl. In some embodiments, a volume of a liquid formulation described herein is administered to the vitreous that is less than about 10 μl. In some embodiments, a volume of a liquid formulation described herein is administered to the vitreous of a subject's eye, including but not limited to a human subject's eye that is between about 0.1 μl and about 200 μl, between about 50 μl and about 200 μl, between about 50 μl and about 150 μl, between about 0.1 μl and about 100 μl, between about 0.1 μl and about 50 μl, between about 1 μl and about 40 μl, between about 1 μl and about 30 μl, between about 1 μl and about 20 μl, between about 1 μl and about 10 μl, or between about 1 μl and about 5 μl. In some embodiments, a volume of a liquid formulation described herein is administered to the vitreous of a subject's eye, including but not limited to a human subject's eye that is between about 1 μl and about 10 μl. In some embodiments, a volume of a liquid formulation described herein is administered to the vitreous of a subject's eye, including but not limited to a human subject's eye that is between about 1 μl and about 5 μl. In some embodiments, a volume of a liquid formulation described herein is administered to the vitreous of a subject's eye, including but not limited to a human subject's eye that is between about 0.1 μl and about 200 μl.

In some embodiments, a volume of a liquid formulation described herein is administered to the vitreous of a subject's eye, including but not limited to a human subject's eye that is any of about 2 μl, about 4 μl, about 5 μl, about 6 μl, about 8 μl, about 10 μl, about 12 μl, about 14 μl, about 16 μl, about 18 μl, or about 20 μl. In some embodiments, a volume of a liquid formulation described herein is administered to the vitreous of a subject's eye, including but not limited to a human subject's eye that is about 2 μl. In some embodiments, a volume of a liquid formulation described herein is administered to the vitreous of a subject's eye, including but not limited to a human subject's eye that is about 5 μl. In some embodiments, a volume of a liquid formulation described herein is administered to the vitreous of a subject's eye that is about 8 μl.

In some embodiments, a total amount of combretastatin administered intravitreally is any of between about 20 μg and about 750 μg, between about 20 μg and about 500 μg, or between about 30 μg and about 200 μg. In some embodiments, a total amount of combretastatin administered intravitreally is any of about 44 μg, about 110 μg, about 132 μg, about 133.5 μg, about 176 μg, about 264 μg, about 352 μg, about 440 μg or about 976 μg. In some embodiments, a total amount of combretastatin administered intravitreally is about 44 μg. In some embodiments, a total amount of combretastatin administered intravitreally is about 110 μg. In some embodiments, a total amount of combretastatin administered intravitreally is about 967 μg. In some embodiments, a liquid formulation containing an amount of combretastatin of 44 μg is intravitreally administered to a human subject by administering about 2 μl of a liquid formulation described herein. In some embodiments, a liquid formulation containing an amount of combretastatin of 110 μg is intravitreally administered to a human subject by administering about 5 μl of a liquid formulation described herein. In some embodiments, a liquid formulation containing an amount of combretastatin of 176 μg is intravitreally administered to a human subject by administering about 8 μl of a liquid formulation described herein.

In some embodiments, a total volume of a liquid formulation described herein is subconjunctivally administered to a subject's eye, including but not limited to a human subject's eye that is less than about 1000 μl, less than about 900 μl, less than about 800 μl, less than about 700 μl, less than about 600 μl, less than about 500 μl, less than about 400 μl, less than about 300 μl, less than about 200 μl, less than about 100 μl, less than about 90 μl, less than about 80 μl, less than about 70 μl, less than about 60 μl, less than about 50 μl, less than about 40 μl, less than about 30 μl, less than about 20 μl, less than about 10 μl, less than about 5 μl, less than about 3 μl, or less than about 1 μl. In some embodiments, a volume of a liquid formulation described herein is subconjunctivally administered to a subject's eye, including but not limited to a human subject's eye that is less than about 20 μl. In some embodiments, a volume of a liquid formulation described herein is subconjunctivally administered to a subject's eye, including but not limited to a human subject's eye that is less than about 10 μl. In some embodiments, a volume of a liquid formulation described herein is subconjunctivally administered to a subject's eye, including but not limited to a human subject's eye that is between about 0.11 and about 200 μl, between about 50 μl and about 200 μl, between about 200 μl and about 300 μl, between about 300 μl and about 400 μl, between about 400 μl and about 500 μl, between about 600 μl and about 700 μl, between about 700 μl and about 800 μl, between about 800 μl and about 900 μl, between about 900 μl and about 1000 μl, between about 50 μl and about 150 μl, between about 0.1 μl and about 100 μl, between about 0.1 μl and about 50 μl, between about 1 μl and about 40 μl, between about 1 μl and about 30 μl, between about 1 μl and about 20 μl, between about 1 μl and about 10 μl, or between about 1 μl and about 5 μl. In some embodiments, a volume of a liquid formulation described herein is subconjunctivally administered to a subject's eye, including but not limited to a human subject's eye that is between about 1 μl and about 10 μl. In some embodiments, a volume of a liquid formulation described herein is subconjunctivally administered to a subject's eye, including but not limited to a human subject's eye that is between about 1 μl and about 5 μl. In some embodiments, a volume of a liquid formulation described herein is administered to subconjunctivally administered to a subject's eye, including but not limited to a human subject's eye that is between about 1 μl and about 5 μl.

In some embodiments, a volume of a liquid formulation described herein is administered to subconjunctivally administered to a subject's eye, including but not limited to a human subject's eye that is between about 0.1 μl and about 200 μl.

In some embodiments, a total volume of a liquid formulation is subconjunctivally administered to a subject's eye, including but not limited to a human subject's eye that is less than about 150 μl. In some embodiments, a volume of a liquid formulation described herein is subconjunctivally administered to a subject's eye, including but not limited to a human subject's eye that is any of about 10 μl, about 20 μl, about 30 μl, about 40 μl, about 50 μl, about 60 μl, about 70 μl, about 80 μl, about 90 μl, or about 100 μl. In some embodiments, a volume of a liquid formulation described herein is subconjunctivally administered to a subject's eye, including but not limited to a human subject's eye that is about 10 μl. In some embodiments, a volume of a liquid formulation described herein is subconjunctivally administered to a subject's eye, including but not limited to a human subject's eye that is about 20 μl. In some embodiments, a volume of a liquid formulation described herein is subconjunctivally administered to a subject's eye, including but not limited to a human subject's eye that is about 30 μl. In some embodiments, a volume of a liquid formulation described herein is subconjunctivally administered to a subject's eye, including but not limited to a human subject's eye that is 40 μl. In some embodiments, a volume of a liquid formulation described herein is subconjunctivally administered to a subject's eye, including but not limited to a human subject's eye that is any of between about 10 μl and about 50 μl, between about 15 μl and about 45 μl, between about 20 μl and about 40 μl, or between about 25 μl and about 35 μl.

In some embodiments, a total amount of combretastatin administered subconjunctivally is any of between about 50 μl and about 3 μl, between about 150 μl and about 750 μl, between about 300 μl and about 1000 μl, between about 300 μl and about 950 μl, between about 400 μl and about 900 μl, between about 450 μl and about 850 μl, between about 500 μl and about 800 μl, between about 550 μl and about 750 μl, or between about 600 μl and about 700 μl. In some embodiments, a total amount of combretastatin administered subconjunctivally is any of about 220 μl, about 440 μl, about 587 μl, about 630 μl, about 660 μl, about 880 μl, about 1320 μl, about 1760 μl, or about 2200 μl. In some embodiments, a total amount of combretastatin administered subconjunctivally is about 220 μl. In some embodiments, a total amount of combretastatin administered subconjunctivally is about 440 μl. In some embodiments, a total amount of combretastatin administered subconjunctivally is about 660 μg. In some embodiments, a total amount of combretastatin administered subconjunctivally is about 880 μl. In some embodiments, a liquid formulation containing an amount of combretastatin of 220 μl is subconjunctivally administered to a human subject by administering about 10 μl of a liquid formulation described herein. In some embodiments, a liquid formulation containing an amount of combretastatin of 440 μl is subconjunctivally administered to a human subject by administering about 20 μl of a liquid formulation described herein. In some embodiments, a liquid formulation containing an amount of combretastatin of 660 μl is subconjunctivally administered to a human subject by administering about 30 μl of a liquid formulation described herein. In some embodiments, a liquid formulation containing an amount of combretastatin of 880 μl is subconjunctivally administered to a human subject by administering about 40 μl of a liquid formulation described herein.

In some embodiments, a total volume of a liquid formulation described herein is subtenonally administered to a subject's eye, including but not limited to a human subject's eye that is less than about 1000 μl, less than about 900 μl, less than about 800 μl, less than about 700 μl, less than about 600 μl, less than about 500 μl, less than about 400 μl, less than about 300 μl, less than about 200 μl, less than about 150 μl, less than about 100 μl, less than about 90 μl, less than about 80 μl, less than about 70 μl, less than about 60 μl, less than about 50 μl, less than about 40 μl, less than about 30 μl, less than about 20 μl, less than about 10 μl, less than about 5 μl, less than about 3 μl, or less than about 1 μl. In some embodiments, a volume of a liquid formulation described herein is subtenonally administered to a subject's eye, including but not limited to a human subject's eye that is less than about 200 μl. In some embodiments, a volume of a liquid formulation described herein is subtenonally administered to a subject's eye, including but not limited to a human subject's eye that is less than about 100 μl. In some embodiments, a volume of a liquid formulation described herein is subtenonally administered to a subject's eye, including but not limited to a human subject's eye that is between about 0.1 μl and about 200 μl, between about 50 μl and about 200 μl, between about 200 μl and about 300 μl, between about 300 μl and about 400 μl, between about 400 μl and about 500 μl, between about 600 μl and about 700 μl, between about 700 μl and about 800 μl, between about 800 μl and about 900 μl, between about 900 μl and about 1000 μl, between about 50 μl and about 150 μl, between about 0.1 μl and about 100 μl, between about 0.1 μl and about 50 μl, between about 1 μl and about 40 μl, between about 1 μl and about 30 μl, between about 1 μl and about 20 μl, between about 1 μl and about 10 μl, or between about 1 μl and about 5 μl. In some embodiments, a volume of a liquid formulation described herein is subtenonally administered to a subject's eye, including but not limited to a human subject's eye that is between about 10 μl and about 200 μl. In some embodiments, a volume of a liquid formulation described herein is administered subtenonally to a subject's eye, including but not limited to a human subject's eye that is between about 0.1 μl and about 200 μl. In some embodiments, a volume of a liquid formulation described herein is subtenonally administered to a subject's eye, including but not limited to a human subject's eye that is between about 50 μl and about 150 μl. In some embodiments, a volume of a liquid formulation described herein is subtenonally administered to a subject's eye, including but not limited to a human subject's eye that is about 30 μl. In some embodiments, a volume of a liquid formulation described herein is subtenonally administered to a subject's eye, including but not limited to a human subject's eye that is about 120 μl.

In some embodiments, a total amount of combretastatin less than about 5 mg is administered subtenonally. In some embodiments, a total amount of combretastatin less than about 5.0 mg is administered subtenonally. In some embodiments, a total amount of combretastatin less than about 4.5 mg is administered subtenonally. In some embodiments, a total amount of combretastatin less than about 4.0 mg is administered subtenonally. In some embodiments, a total amount of combretastatin less than about 3.5 mg is administered subtenonally. In some embodiments, a total amount of combretastatin less than about 3.0 mg is administered subtenonally. In some embodiments, a total amount of combretastatin less than about 2.5 mg is administered subtenonally. In some embodiments, a total amount of combretastatin less than about 2 mg is administered subtenonally. In some embodiments, a total amount of combretastatin less than about 1.2 mg is administered subtenonally. In some embodiments, a total amount of combretastatin less than about 1.0 mg is administered subtenonally. In some embodiments, a total amount of combretastatin less than about 0.8 mg is administered subtenonally. In some embodiments, a total amount of combretastatin less than about 0.6 mg is administered subtenonally. In some embodiments, a total amount of combretastatin less than about 0.4 mg is administered subtenonally. In some embodiments, a volume of a formulation is administered that contains an amount of combretastatin described herein.

In some embodiments, the combretastatin may be combretastatin A-4 or an analog, prodrug or derivative thereof. In some implementations, the combretastatin is combretastatin A-4 phosphate.

“Total amount of a combretastatin” as used herein refers to the total amount of a combretastatin administered during a single administration session by a patient and/or physician and/or other medical professional. In some embodiments, a single administration session will involve a single administration of the combretastatin. In some embodiments, one administration session will include more than one administration of the combretastatin. In some embodiments, one administration session will include a single route of administration.

In some embodiments, one administration session will include multiple routes of administration. Thus, in some embodiments, portions of the total amount of the combretastatin are administered separately during a single administration session. In such embodiments, the portions of the total amount that are administered separately may be administered by the same and/or different routes of administration. In addition, in some embodiments, portions of the total amount that are administered separately may be administered in the same and/or different formulations.

“Total volume of a liquid formulation” as used herein refers to the total volume of a liquid formulation administered during a single administration session by a patient and/or physician and/or other medical professional. In some embodiments, a single administration session will involve a single administration of the liquid formulation. In some embodiments, one administration session will include more than one administration of the liquid formulation. In some embodiments, one administration session will include a single route of administration. In some embodiments, one administration session will include multiple, different routes of administration. Thus, in some embodiments, portions of the total volume are administered separately during a single administration session. In such embodiments, the portions of the total volume that are administered separately may be administered by the same and/or by different routes of administration.

In some embodiments, a combretastatin is administered in multiple ocular locations. In some embodiments, a combretastatin is administered intravitreally and subconjunctivally. In some embodiments, a combretastatin is first administered intravitreally and at least one subsequent administration is administered subconjunctivally. In some embodiments, a combretastatin is first administered subconjunctivally and at least one subsequent administration is administered intravitreally. In some embodiments, a combretastatin is first administered intravitreally, subconjunctivally, or subtenonally and at least on subsequent administration is topically. In some embodiments, the same (i.e., identical) liquid formulation of the combretastatin is used for the first and subsequent administrations. In some embodiments, a different liquid formulation is used for the first and subsequent administrations. In some embodiments, administration to multiple ocular locations occurs during one visit to the physician. In some embodiments, administration to multiple ocular locations occurs during separate visits to the physician.

In some embodiments of the liquid formulations described herein, the combretastatin is a solution or suspension of CA4P in a liquid medium. The liquid formulations described herein may comprise a solubilizing agent component. In some embodiments the solubilizing agent component is a surfactant. Note that there is some overlap between components that may be solvents and solubilizing agents, and therefore the same component may in some systems be used as either a solvent or a solubilizing agent. A liquid formulation that comprises a combretastatin and a component that may be considered either a solvent or a solubilizing agent or surfactant will be considered a solvent if it is playing the role of a solvent; if the component is not playing the role of the solvent, the component may be considered a solubilizing agent or surfactant.

Generally, any solubilizing agent or combination of solubilizing agents may be used in the liquid formulations described herein. In some embodiments, the solubilizing agent is a surfactant or combination of surfactants. Many surfactants are possible. Combinations of surfactants, including combinations of various types of surfactants, may also be used. For instance, surfactants which are nonionic, anionic (i.e. soaps, sulfonates), cationic (i.e. CTAB), zwitterionic, polymeric or amphoteric may be used.

Surfactants that can be used may be determined by mixing a combretastatin of interest with a putative solvent and a putative surfactant, and observing the characteristics of the formulation after exposure to a medium. Examples of surfactants include but are not limited to fatty acid esters or amides or ether analogues, or hydrophilic derivatives thereof; monoesters or diesters, or hydrophilic derivatives thereof; or mixtures thereof; monoglycerides or diglycerides, or hydrophilic derivatives thereof; or mixtures thereof; mixtures having enriched mono- or/and diglycerides, or hydrophilic derivatives thereof; surfactants with a partially derivatized with a hydrophilic moiety; monoesters or diesters or multiple-esters of other alcohols, polyols, saccharides or oligosaccharides or polysaccharides, oxyalkylene oligomers or polymers or block polymers, or hydrophilic derivatives thereof, or the amide analogues thereof; fatty acid derivatives of amines, polyamines, polyimines, aminoalcohols, aminosugars, hydroxyalkylamines, hydroxypolyimines, peptides, polypeptides, or the ether analogues thereof.

Liquid formulations may optionally further comprise stabilizers, excipients, gelling agents, adjuvants, antioxidants, and/or other components as described herein.

In some embodiments all components in the liquid formulation, other than the combretastatin, are liquid at room temperature.

In some embodiments, the liquid formulation comprises a release modifying agent. In some embodiments, the release modifying agent is a film-forming polymer component. The film-forming polymer component may comprise one or more film-forming polymers. Any film-forming polymer may be used in the excipient component. In some embodiments, the film-forming polymer component comprises a water insoluble film forming polymer. In some embodiments, the release modifying agent component comprises an acrylic polymer, including but not limited to polymethacrylate, including but not limited to Eudragit RL.

In some embodiments the combretastatin in the liquid formulation comprises between about 0.01 to about 30% of the total weight of the composition; between about 0.05 to about 15%; between about 0.1 to about 10%; between about 1 to about 5%; or between about 5 to about 15%; between about 8 to about 10%; between about 0.01 to about 1%; between about 0.05 to about 5%; between about 0.1 to about 0.2%; between about 0.2 to about 0.3%; between about 0.3 to about 0.4%; between about 0.4 to about 0.5%; between about 0.5 to about 0.6%; between about 0.6 to about 0.7%; between about 0.7 to about 1%; between about 1 to about 5%; between about 5 to about 10%; between about 15 to about 30%, between about 20 to about 30%; or between about 25 to about 30%.

In some embodiments the combretastatin in the liquid formulation comprises between about 0.001 to about 1.00% of the total weight of the composition. In some embodiments the combretastatin in the liquid formulation comprises any of about 0.07%, about 0.08%, 0.09%, 0.17%, 1.38%, 1.47%, 2%, 4%, 4.84%, or 5% of the total weight of the composition. In some embodiments, the combretastatin may be a combretastatin compound. In some embodiments, the combretastatin may be CA4 or an analog, prodrug or derivative thereof. In some embodiments, the combretastatin is CA4P.

The solvent component of the liquid formulation may comprise, for instance, between about 0.01 to about 99.9% of the total weight of the composition; between about 0.1 to about 99%; between about 25 to about 55%; between about 30 to about 50%; or between about 35 to about 45%; between about 0.1 to about 10%; between about 10 to about 20%; between about 20 to about 30%; between about 30 to about 40%; between about 40 to about 45%; between about 40 to about 45%; between about 45 to about 50%; between about 50 to about 60%; between about 50 to about 70%; between about 70 to about 80%; between about 80 to about 90%; or between about 90 to about 100%.

The solubilizing agent component of the liquid formulation may comprise, for instance, between about 0.01 to about 30% of the total weight of the composition; between about 0.1 to about 20%; between about 2.5 to about 15%; between about 10 to about 15%; or between about 5 to about 10%; between about 8 to about 12%; between about 10 to about 20%; between about 20 to about 30%.

In some embodiments, the liquid formulations described herein have a viscosity of between 40% and 120% centipoise. In some embodiments the liquid formulations described herein have a viscosity of between 60% and 80% centipoise.

The liquid formulations described herein may be administered with or further comprise a viscosity modifying agent. One exemplary viscosity modifying agent that may be used is hyaluronic acid. Hyaluronic acid is a glycosaminoglycan. It is made of a repetitive sequence of glucuronic acid and glucosamine. Hyaluronic acid is present in many tissues and organs of the body, and contributes to the viscosity and consistency of such tissues and organs. Hyaluronic acid is present in the eye, including the vitreous of the eye, and along with collagen contributes to the viscosity thereof. The liquid formulations described herein may further comprise or be administered with hyaluronic acid. Other nonlimiting examples of viscosity modifying agents include polyalkylene oxides, glycerol, carboxymethyl cellulose, sodium alginate, chitosan, dextran, dextran sulfate and collagen. These viscosity modifying agents can be chemically modified.

In some embodiments the liquid formulations described herein comprise a combretastatin and a solvent component. The solvent component may comprise a single solvent or a combination of solvents. In some embodiments, the solvent is glycerin, N-methylpyrrolidone (NMP), dimethyl acetamide (DMA), dimethyl formamide, dimethyl sulfoxide (DMSO), glycerol formal, ethoxy diglycol, triethylene glycol dimethyl ether, triacetin, diacetin, corn oil, acetyl triethyl citrate (ATC), ethyl lactate, polyglycolated capryl glyceride, γ butyrolactone, dimethyl isosorbide, benzyl alcohol, ethanol, isopropyl alcohol, propylene glycol (PG), polyethylene glycol of various molecular weights, including but not limited to PEG 300 and PEG 40 μl, or a mixture of one or more thereof.

In some embodiments, the solvent is polyethoxylated castor oil (e.g., Cremophor (PEG 35 castor oil)), monoglycerides and/or diglycerides of caprylic acid (e.g., Capmul MCM (C8)), nonionic polymer of the alkyl aryl polyether alcohol (e.g., tyloxapol (ethoxylated p-tert-octylphenol formaldehyde polymer)), 50% phosphatidylcholine in propylene glycol/ethanol carrier (e.g., Phosal® 50PG), propylene glycol monolaurate, propylene glycol dicaprylate/dicaprate, macrogol 15 hydroxystearate, ethanol, or a mixture of one or more thereof. In some variation, the solvent may comprise a combination of at least two solvents. In some embodiments, the at least two solvents comprising a first solvent such as polyethoxylated castor oil (e.g., Cremophor (PEG 35 castor oil)), propylene glycol monolaurate, propylene glycol dicaprylate/dicaprate, macrogol 15 hydroxystearate, or nonionic polymer of the alkyl aryl polyether alcohol (e.g., tyloxapol (ethoxylated p-tert-octylphenol formaldehyde polymer)) and a second solvent such as monoglycerides and/or diglycerides of caprylic acid (e.g., Capmul MCM (C8)). In some embodiments, the solvent may further comprise ethanol. In some embodiments, the solvent may further comprise water. In some embodiments, liquid formulation is topically administered. In some embodiments, the liquid formulation is administered as eye drops. In some embodiments, the liquid formulation is used to treat or prevent PCO.

The solvent component may comprise water, for instance, between about 0.01 to about 99.9% of the total weight of the composition; between about 0.1 to about 99%; between about 25 to about 55%; between about 30 to about 50%; or between about 35 to about 45%; between about 0.1 to about 10%; between about 10 to about 20%; between about 20 to about 30%; between about 30 to about 40%; between about 40 to about 45%; between about 40 to about 45%; between about 45 to about 50%; between about 50 to about 60%; between about 50 to about 70%; between about 70 to about 80%; between about 80 to about 90%; or between about 90 to about 100%. In some embodiments, water comprises between about 15 to about 30% (w/w) of the liquid formulation. In some embodiments, water comprises between about 80 to about 90% (w/w) of the liquid formulation. In some embodiments, water comprises at least 15% (w/w). In some embodiments, water comprises at least 20% (w/w) of the liquid formulation. In some embodiments, water comprises at least 25% (w/w) of the liquid formulation. In some embodiments, water comprises at least about 5 percent (w/w) of the liquid formulation. In some embodiments, water comprises about 16% (w/w) of the liquid formulation. In some embodiments, water comprises about 22% (w/w). In some embodiments, water comprises about 28% (w/w) of the liquid formulation. In some embodiments, water comprises about 83% (w/w) of the liquid formulation.

In some embodiments, the liquid formulations described herein are suspensions, and comprise a combretastatin and a diluent component. In some embodiments, the diluent component comprises one or more components listed herein as solvents or solubilizing agents, wherein the resulting mixture is a suspension. In some embodiments the liquid formulation is partly a solution and partly a suspension.

The compositions and formulations described herein may be used to deliver amounts of the combretastatins effective for treating, preventing, or inhibiting PCO. In some embodiments the compositions and formulations described herein deliver one or more vascular disrupting and/or additional active agents over an extended period of time.

An “effective amount,” which is also referred to herein as a “therapeutically effective amount,” of a combretastatin for administration as described herein is that amount of the combretastatin that provides the therapeutic effect sought when administered to the subject, including but not limited to a human subject. The achieving of different therapeutic effects may require different effective amounts of combretastatin. For example, the therapeutically effective amount of a combretastatin used for preventing a disease or condition may be different from the therapeutically effective amount used for treating or inhibiting the disease or condition. In addition, the therapeutically effective amount may depend on the age, weight, and other health conditions of the subject as is well know to those versed in the disease or condition being addressed. Further, the therapeutically effective amount can depend upon the route of administration. Thus, the therapeutically effective amount may not be the same in every subject to which the combretastatin is administered.

An effective amount of a combretastatin for treating, preventing, or inhibiting PCO is also referred to herein as the amount of combretastatin effective to treat, prevent, or inhibit PCO or a symptom thereof.

To determine whether a level of combretastatin is a “therapeutically effective amount” to treat, prevent, or inhibit PCO, formulations may be administered in animal models for PCO, and the effects may be observed. In addition, dose ranging human clinical trials may be conducted to determine the therapeutically effective amount of a combretastatin.

Generally, the combretastatin may be formulated in any composition or formulation capable of delivery of a therapeutically effective amount of the combretastatin to a subject or to the eye of a subject for the required delivery period. Compositions include liquid formulations.

In some embodiments, the formulations described herein are provided in one or more unit dose forms, wherein the unit dose form contains an amount of a liquid formulation described herein that is effective to treat or prevent the disease or condition for which it is being administered. In some embodiments, the formulations described herein are provided in one or more unit dose forms, wherein the unit dose form contains an amount of a liquid combretastatin formulation described herein that is effective to treat or prevent PCO.

In some embodiments, the unit dose form is prepared in the concentration at which it will be administered. In some embodiments, the unit dose form is diluted prior to administration to a subject. In some embodiments, a liquid formulation described herein is diluted in an aqueous medium prior to administration to a subject. In some embodiments the aqueous medium is an isotonic medium. In some embodiments, a liquid formulation described herein is diluted in a non-aqueous medium prior to administration to a subject.

In a further aspect, provided herein are kits comprising one or more unit dose forms as described herein. In some embodiments, the kit comprises one or more of packaging and instructions for use to treat one or more diseases or conditions. In some embodiments, the kit comprises a diluent which is not in physical contact with the formulation or pharmaceutical formulation. In some embodiments, the kit comprises any of one or more unit dose forms described herein in one or more sealed vessels. In some embodiments, the kit comprises any of one or more sterile unit dose forms.

In some embodiments, the unit dose form is in a container, including but not limited to a sterile sealed container. In some embodiments the container is a vial, ampule, or low volume applicator, including but not limited to a syringe. In some embodiments, a low-volume applicator is pre-filled with combretastatin, including but not limited to CA4P, for treatment of PCO. Described herein is a pre-filled low-volume applicator pre-filled with a formulation comprising a combretastatin, including but not limited to CA4P. In some embodiments a low-volume applicator is pre-filled with a solution comprising a combretastatin, including but not limited to CA4P, and optionally further comprises one or more additional components, including but nor limited to a solvent, surfactant and/or a stabilizer.

Described herein are kits comprising one or more containers. In some embodiments a kit comprises one or more low-volume applicators is pre-filled with a formulation described herein comprising a combretastatin, including but not limited to formulations comprising CA4P, and optionally further comprises one or more additional components. In some embodiments the kit comprises one or more containers, including but not limited to pre-filled low-volume applicators, with instructions for its use. In a further variation a kit comprises one or more low-volume applicators pre-filled with CA4P, with instructions for its use in treating PCO. In some embodiments, the containers described herein are in a secondary packaging.

### V. EXAMPLES

Combretastatin-A-4-phosphate (CA4P) is an inhibitor of microtubule polymerization that has been shown to disrupt vasculature within tumors (Quan et al., Int. J. Cancer, 122: 1730-1737 (2008)). We tested CA4P as a potential inhibitor of lens epithelial cells epithelial-mesenchymal transition (EMT) during posterior capsular opacification (PCO), a common complication of cataract surgery. As demonstrated by anti-tubulin immunofluorescence and immunoblotting, a 30 minute exposure to 1 μM CA4P was sufficient to effectively depolymerize microtubule networks in both in vitro cultured immortalized bovine lens epithelial cells and primary bovine lens epithelial cells grown in vitro on their natural basement membrane, the lens capsule. This effect persisted up to 48 h after CA4P exposure. Lens epithelial cell proliferation was also affected, as assayed by Ki67 immunofluorescence, MTT-based cell viability assays and cell cycle analysis by flow cytometry. Hallmarks of EMT, namely loss of cell polarity and migratory behavior, were also investigated in primary bovine lens epithelial cells grown on the lens capsule and were decreased by CA4P exposure, although cell multilayering seemed unaffected. These results identify a potential novel therapeutic application of an already well-characterized compound, with significant effectiveness being achieved for up to two days after a single 30-minute exposure.

### A. Example 1

### CA4P Inhibits Lens Epithelial Cell Proliferation

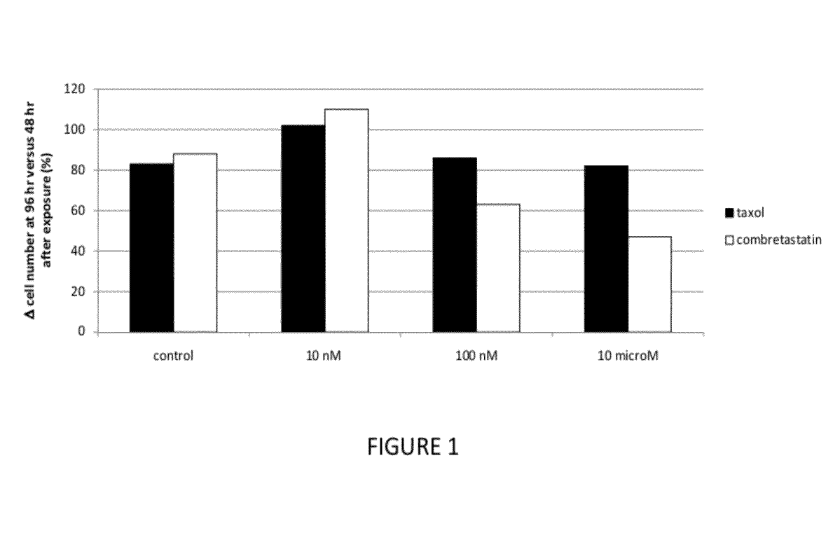

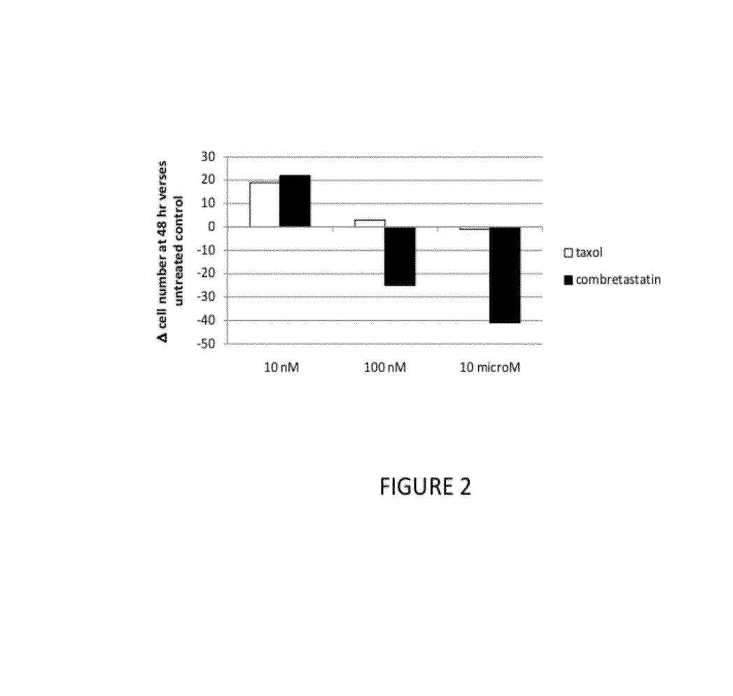

Primary bovine lens epithelial cells were cultured in serum-free conditions, survival factors being released from the lens capsule. Paclitaxel or combretastatin A-4 phosphate was added to the culture to a final concentration of 10 nM, 100 nM or 10 μM. Cell number was determined for each capsule at 48 h and 96 h after addition of the test agent. Two capsules were taken per treatment and cell numbers averaged after being expressed as a percentage of 48 h value. This indicates the increase/decrease in cell number within a 48 h time period (i.e. from 48 h to 96 h), normalized as a percentage. Independent T-test P value between CA4P control and 10 μM is 0.0002. Combretastatin A-4 phosphate is more effective than paclitaxel at inducing a decline in cell number over a 96 hour exposure (FIG. 1 and FIG. 2).

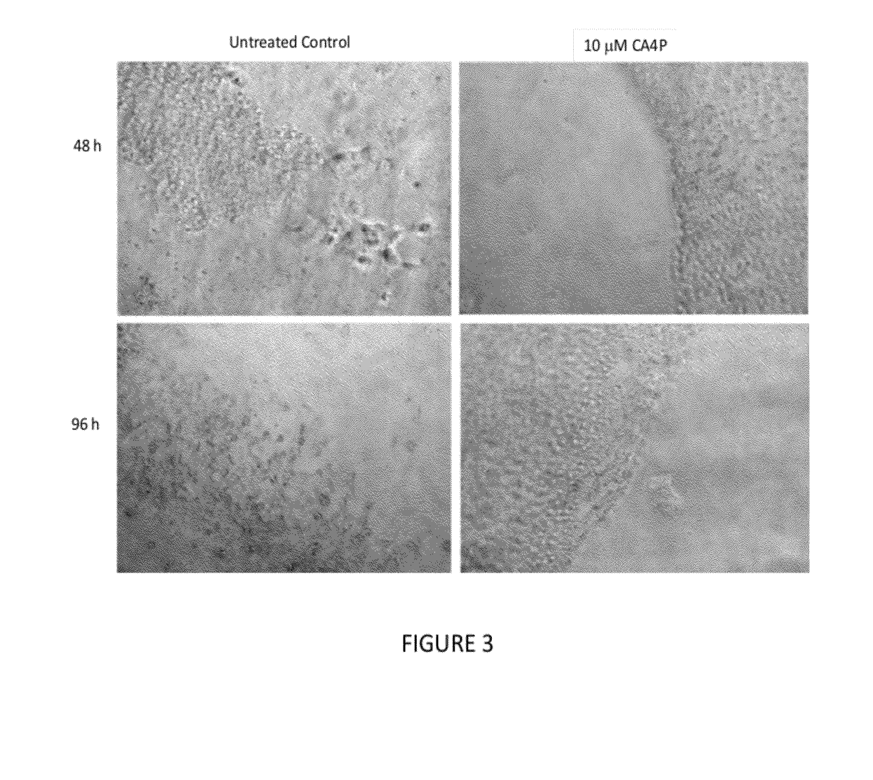

By light microscopy, CA4P has a dramatic effect upon cell morphology, retaining the cobblestone characteristic of the lens epithelium, when in the control cultures the lens cells have begun to trans-differentiate actinₛₘ/desmin positive cells and migrate off the capsule. Pictures were taken from live primary bovine cells on the lens capsule with FujiFinepix (FIG. 3). The untreated controls show a mix of epithelial cells sheets and fibroblast-like cells clusters, while the treated samples (CA4P 10 μM) show only epithelial sheets with the typical cobblestone appearance of the lens epithelium.

### B. Example 2

### Transient Exposure to CA4P

1. Loss of Tubulin Polymers in Cultured Cells

BMI-immortalized bovine lens epithelial (BIE) cells, on plastic, and primary bovine lens epithelial cells, on their original in vivo basement membrane, (the eye lens capsule), were cultured in 10% fetal calf serum (FCS)-supplemented medium at 37° C. prior to 30 min exposure to 1 μM CA4P, after which they were returned to 10% FCS-supplemented medium. The relatively short exposure was chosen to approximate the duration of a cataract surgery procedure. At a range of time points following exposure, cells were subjected to cell viability assay (MTT-based assay, PROMEGA, UK), immunoblotting or immunofluorescence.

BMI-immortalized bovine lens epithelial cells (BIE cell line, produced in Roy Quinlan's Laboratory, University of Durham, UK), were grown at 37° C., 5% CO₂, in 10 cm diameter Petri dishes in 10% fetal calf serum (FCS)-supplemented tissue culture medium (Sigma-Aldrich, UK). When nearing confluence, they were exposed to 1 μM combretastatin A 4-phosphate (CA4P) (OXiGENE, UK) diluted in 10% FCS-supplemented medium for 30 min, after which the cells were returned to 10% FCS-supplemented medium. Immediately after the end of CA4P exposure, cells were extracted to assess the proportion of dimer/polymer tubulin, as described in Lieuvin et al., 1994 (J. Cell Biology 124 (6):985-996). Briefly, cells were washed twice in phosphate buffered saline (PBS), then extracted for 3 min in 5004 lysis buffer (80 mM Pipes-KOH pH 6.8, 1 mM MgCl₂, 1 mM EGTA, 0.5% Triton X-100 (vol/vol), 10% glycerol (vol/vol)), that had been warmed to 30° C. beforehand. The cell lysate was left to collect at the edge of the Petri dish without scraping the cells and added to 5004 of 2× sample buffer (100 mM Tris.HCl pH 6.8, 2 mM EDTA, 2% SDS, 40% glycerol (vol/vol)), before being boiled for 3 min. This first extract contains the dimer tubulin fraction. The cells, having remained in the Petri dish, were washed once in 10 mL lysis buffer for 30 min, after which they were extracted in 500 μL 1% SDS for 3 min. The cells were then scraped and this second lysate was added to 500 μLof 2× sample buffer, followed by syringing (25G needle, BD Microlance, Ireland) and boiling for 3 min. This second extract contains the polymer tubulin fraction. This procedure was repeated at 24 h after the end of CA4P exposure. All extracts were kept at −20° C. until use.

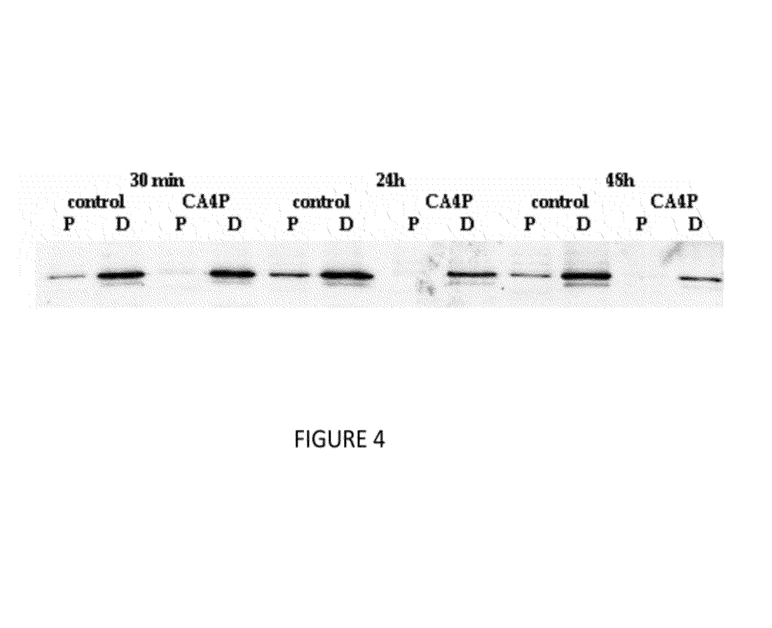

The proportion of dimer/polymer tubulin was assessed by western blotting. 15 μL of extract were loaded per lane of a 12% acrylamide denaturing gel, which was run at 200V for 40 min, followed by blotting onto a nitrocellulose membrane at 0.8 mA/cm² for 2 h and blocking in either milk (α-tubulin) or bovine serum albumin (GAPDH) in Tween-Tris buffered saline (TTBS: 0.2% Tween (vol/vol), 150 mM NaCl, 20 mM Tris.HCl pH 7.4) for 1 h at room temperature. After a single 5 min wash in TTBS, the membrane was then exposed to primary antibody (α-tubulin: mouse monoclonal, 1/1000, gift from Dr. Karakesisoglou, University of Durham, UK; GAPDH: mouse monoclonal, 1/1000, ab9484 (ABCAM, UK)) in TTBS overnight at 4° C. The following morning, the membrane was washed three times 10 min in TTBS, then exposed to secondary antibody (polyclonal goat anti-mouse horseradish peroxidase (HRP)-conjugated, 1/1000, DAKO, Denmark) for 1 h at 37° C. After another three 10 min washes in TTBS, the membrane was developed by chemiluminescence (20 ml of 1:1 mix of buffer 1 (100 mM Tris.HCl pH 8.5, 0.02% H₂O₂) and buffer 2 (100 mM Tris.HCl pH 8.5, 0.44% coumaric acid, 1% luminol) for 5 min in the dark, after which the signal was detected on a FujiFilm Intelligent Dark Box 11 and analyzed via IR-LAS-1000 Pro V3.12 software. A single 30 min treatment with 1 μM CA4P results in the loss of polymerized tubulin both at 30 min after the start of CA4P exposure (i.e. immediately after the end of exposure) and up to 24 h after the end of CA4P exposure (FIG. 4).

2. Loss of Microtubules in Primary Bovine Lens Epithelial Cells

Bovine eyes were obtained from Coast and County Meat Group (Felling, UK) on the day of culling and lenses were dissected out of the eyes under a sterile tissue culture hood as described below. After generous dousing in 70% ethanol, eyes were cut open using a major surgical blade (Swann-Morton, UK) via a single cut, parallel and posterior to the cornea-conjunctiva limit. The eye was then inverted and the vitreous removed. The lens was freed from the ciliary fibers and immediately placed anterior face down in a SYLGARD-covered (Dow Corning, Germany) well of a 12-well tissue culture plate (Greiner Bio-One, UK). The posterior side of the lens capsule was cut in a Y-shape fashion by a thin scalpel and the resulting flaps pulled back and pinned onto the SYLGARD. The fiber mass was removed by gently rolling it off the anterior lens capsule, thus leaving most of the primary lens epithelial cell layer attached onto the anterior lens capsule. This primary lens epithelial cell layer was then cultured on its original lens capsule at 37° C., 5% CO₂, in 0% FCS-supplemented tissue culture.

Immediately after dissection, primary lens epithelial cells were exposed for 30 min to 1 mM CA4P diluted in 0% FCS medium, after which they were returned to 0% FCS-supplemented medium. Immediately after the end of the CA4P exposure and 48 h after the end of the CA4P exposure, the presence of polymerized tubulin in the primary lens epithelial cell layers was investigated by immunofluorescence.

Cells on the lens capsule were rinsed three times in PBS, followed by a single 20 min fixation in a 1:1 mix of methanol and acetone, three rinses in PBS/0.2 g/L BSA/0.2 g/L Na Azide and a single 20 min blocking step in 10% goat serum (Sigma-Aldrich, UK) diluted in PBS/BSA/NaAzide. The cells were then exposed to primary antibody (α-tubulin: mouse monoclonal, 1/100, gift from Dr. Karakesisoglou, University of Durham, UK) for 1 h at room temperature, after which they were rinsed three times in PBS/BSA/NaAzide and exposed to secondary antibody (goat anti-mouse IgG FITC-conjugated, 1/100; DAPI, 1/1000 (Sigma-Aldrich, UK)) for 1 h at room temperature in the dark. After three last rinses in PBS/BSA/NaAzide, the cell layers and their lens capsules were plated onto microscope slides (VWR International, Belgium) and examined using a Zeiss LSM 510 Meta confocal microscope and its associated software.

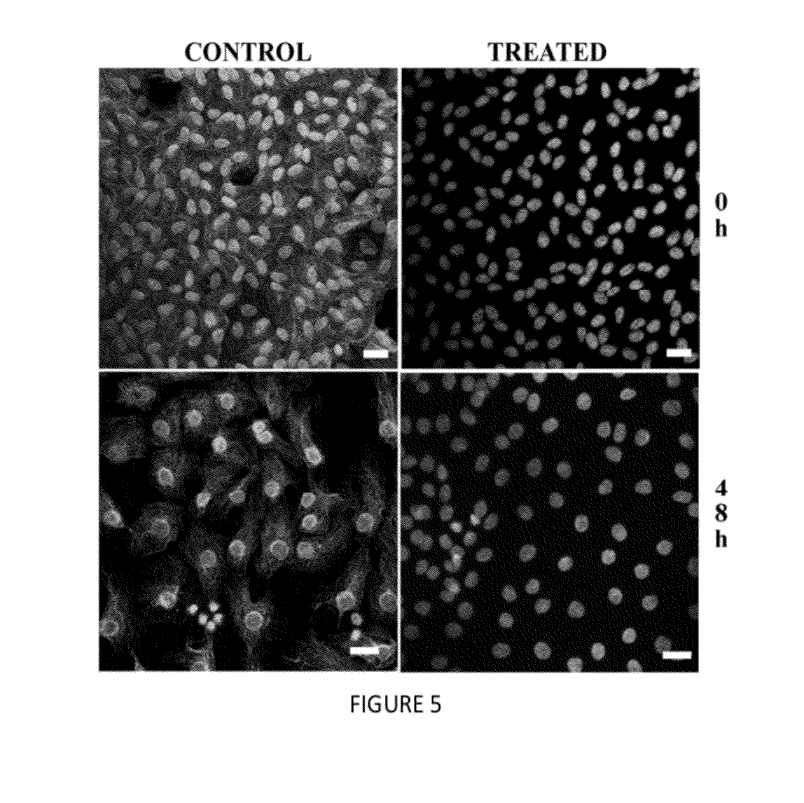

A single 30 min treatment with 1 μM CA4P results in the loss of polymerized tubulin both at 30 min after the start of CA4P exposure (i.e., immediately after the end of exposure) and up to 48 h after the end of CA4P exposure. (FIG. 5). In addition, primary bovine lens epithelial cells grown on the lens capsule in serum-free conditions for 48 h move away from the wound edge. 30 min exposure to 1 mM CA4P immediately after wounding prevents the cells from leaving the wound edge, but does not prevent cell multi-layering (data not shown).

3. Cell viability

BMI-immortalized bovine lens epithelial cells (BIE cell line, produced in Roy Quinlan's Laboratory, University of Durham, UK), were seeded onto 12-well tissue culture plates in 10% fetal calf serum (FCS)-supplemented tissue culture medium (Sigma-Aldrich, UK) and kept at 37° C., 5% CO₂ overnight. The next morning, they were exposed to 1 mM CA4P diluted in 10% FCS-supplemented medium for 30 min, after which the cells were returned to 10% FCS-supplemented medium. Viable cell number was assayed immediately and at 24 h, 48 h and 72 h after the end of exposure, using an MTT-based colorimetric assay (CellTiter95® Aqueous non-radioactive cell proliferation assay, Promega, UK), according to manufacturer's instructions. Briefly, the original cell culture medium was discarded from all wells and replaced by 5004 of fresh medium, to which was added 1004 of kit reagent. The wells were then returned to 37° C., 5% CO₂ for 2 h 30 in the dark, after which 2004 were transferred from each well into a 96-well plate, which was read at 492 nm absorbance on an Anthos Lucy I (Anthos Labtech Instruments, Germany). The absorbance values were converted to cell numbers using standard curves specific to lens epithelial cells (determined in Roy Quinlan's laboratory, University of Durham, UK), and the viable cell numbers obtained were averaged with standard deviation. Significant differences in the values obtained between control and treated cells at different timepoints were assessed using an independent, two-tailed t-test. Figure shows collated results of three independent repeats.

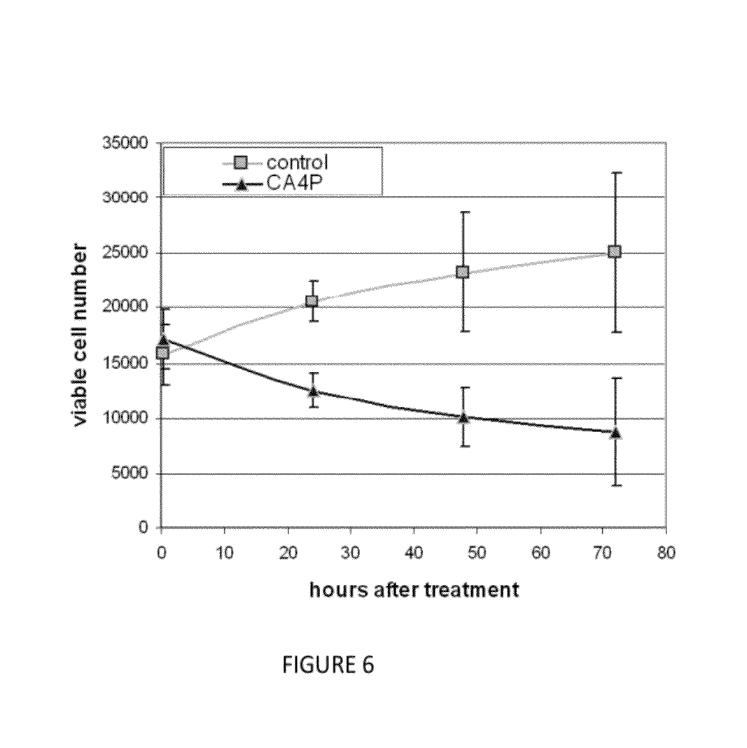

A single 30 min treatment with 1 μM CA4P results in the steady reduction in the number of BIE cells over 72 h following CA4P treatment (FIG. 6).

4. Cell Proliferation

BMI-immortalized bovine lens epithelial cells (BIE cell line, produced in Roy Quinlan's Laboratory, University of Durham, UK), were seeded onto 13 mm round coverslips in 6-well tissue culture plates in 10% fetal calf serum (FCS)-supplemented tissue culture medium (Sigma-Aldrich, UK) and kept at 37° C., 5% CO₂ overnight. The next morning, they were exposed to 1 μM CA4P diluted in 10% FCS-supplemented medium for 30 min, after which the cells were returned to 10% FCS-supplemented medium. After 48 h, the prevalence of Ki67 positive cells was assessed by immunofluorescence.

BIE cells on coverslips were rinsed three times in PBS, followed by a single 20 min fixation in a 1:1 mix of methanol and acetone, three rinses in PBS/0.2 g/L BSA/0.2 g/L Na Azide and a single 20 min blocking step in 10% goat serum (Sigma-Aldrich, UK) diluted in PBS/BSA/NaAzide. The cells were then exposed to primary antibody (Ki-67: mouse monoclonal, 1/100, MIB-1 (DAKO, Denmark)) for 1 h at room temperature, after which they were rinsed three times in PBS/BSA/NaAzide and exposed to secondary antibody (goat anti-mouse IgG FITC-conjugated, 1/100; DAPI, 1/1000 (Sigma-Aldrich, UK)) for 1 h at room temperature in the dark. After three last rinses in PBS/BSA/NaAzide, the cells were plated onto microscope slides (VWR International, Belgium) and examined using a Zeiss LSM 510 Meta confocal microscope and its associated software. Ki-67 positive cells were counted out of a total of between 200 and 500 cells per coverslip, for two coverslips for each of three independent repeats, and expressed as the average percentage of Ki-67 positive cells out of the total number of cells counted (as determined by DAPI staining). Significant differences between control and treated cells were assessed by Chi²-test.

The proportion of Ki67 (associated with cell proliferation) positive cells was similar in both control and treated cells, as determined at 48 h after exposure (Table 1), although changes in nuclear morphology, as well as multinucleated cells were detected 30 h after exposure (data not shown). This abnormal nuclear morphology is reminiscent of defective cytoplasmic separation during mitosis. Notably, by 48 h, cell loss is extensive and no cells were observed that displayed nuclear morphological abnormalities, which suggest that those ‘abnormal’ cells are lost between 30 h and 48 h after CA4P exposure.

TABLE 1

| | Ki67 negative | Ki67 positive |
|---|---|---|
| | (%) | (%) |
| control | 84.9 ± 6.2 | 15.1 ± 6.2 |
| CA4P | 85.1 ± 5.7 | 14.9 ± 5.7 |

5. Erk ½ Phosphorylation

Bovine eyes were obtained from Coast and County Meat Group (Felling, UK) on the day of culling and lenses were dissected out of the eyes under a sterile tissue culture hood as described below. After generous dousing in 70% ethanol, eyes were cut open using a major surgical blade (Swann-Morton, UK) via a single cut, parallel and posterior to the cornea-conjunctiva limit. The eye was then inverted and the vitreous removed. The lens was freed from the ciliary fibers and immediately placed anterior face down in a SYLGARD-covered (Dow Corning, Germany) well of a 12-well tissue culture plate (Greiner Bio-One, UK). The posterior side of the lens capsule was cut in a Y-shape fashion by a thin scalpel and the resulting flaps pulled back and pinned onto the SYLGARD. The fiber mass was removed by gently rolling it off the anterior lens capsule, thus leaving most of the primary lens epithelial cell layer attached onto the anterior lens capsule. This primary lens epithelial cell layer was then cultured on its original lens capsule at 37° C., 5% CO₂, in 0% FCS-supplemented tissue culture.

Immediately after dissection, primary lens epithelial cells were exposed for 30 min to 1 mM CA4P diluted in 0% FCS medium, after which they were returned to 0% FCS-supplemented medium. Immediately after and 1 h after the end of the CA4P exposure, the phosphorylation status of ERK 1/2 (p42 and p44) in the primary lens epithelial cell layers was investigated by immunoblotting. Briefly, cells were washed twice in phosphate buffered saline (PBS), then extracted for 3 min in 1504 of 2× sample buffer (100 mM Tris.HCl pH 6.8, 2 mM EDTA, 2% SDS, 40% glycerol (vol/vol)) per well, before being syringed and boiled for 3 min. All extracts were kept at −20° C. until use. After total protein quantification of the extracts by Bicinchoninic Acid (BCA) assay (ThermoScientific, UK), 20 μg of extract were loaded per lane of a 12% acrylamide denaturing gel, which was run at 200V for 40 min, followed by blotting onto a nitrocellulose membrane at 0.8 mA/cm² for 2 h and blocking in either milk (Erk 1/2) or BSA (GAPDH) in TTBS (0.2% Tween (vol/vol), 150 mM NaCl, 20 mM Tris.HCl pH 7.4) for 1 h at room temperature. After a single 5 min wash in TTBS, the membrane was then exposed to primary antibody (phosphor-Erk ½ (Thr202/Tyr204) (D13.14.4E): rabbit monoclonal, 1/1000, (Cell Signaling Technology, UK); GAPDH: mouse monoclonal, 1/1000, ab9484 (ABCAM, UK)) in TTBS overnight at 4° C. The following morning, the membrane was washed three times 10 min in TTBS, then exposed to secondary antibody (polyclonal goat anti-mouse HRP-conjugated, 1/1000; polyclonal swine anti-rabbit HRP-conjugated (DAKO, Denmark)) in TTBS overnight at 4° C. After another three 10 min washes in TTBS, the membrane was developed by chemiluminescence (20 ml of 1:1 mix of buffer 1 (100 mM Tris.HCl pH 8.5, 0.02% H₂O₂) and buffer 2 (100 mM Tris.HCl pH 8.5, 0.44% coumaric acid, 1% luminol) for 5 min in the dark, after which the signal was detected on a FujiFilm Intelligent Dark Box II and analyzed via IR-LAS-1000 Pro V3.12 software. The phosphorylated levels of Erk ½ were quantified by densitometry using the ImageGauge V4.23 software and normalization to the GAPDH levels.

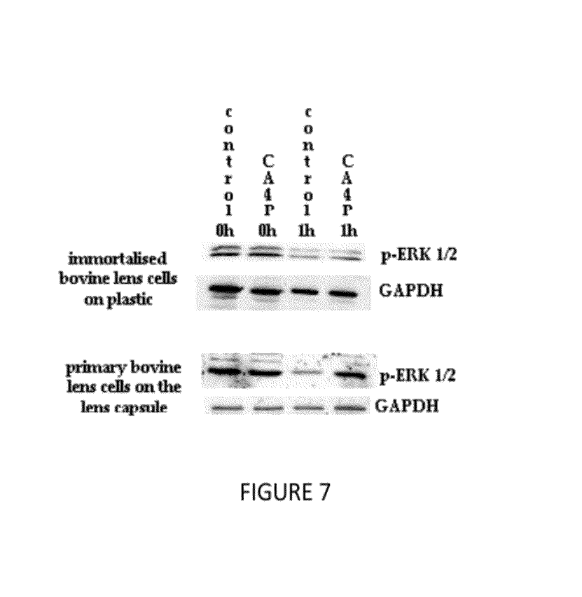

Phospho-Erk ½ levels were elevated immediately after the end of CA4P exposure in both control and treated cells and decreased 1 h after the end of CA4P exposure in both control and treated cells (FIG. 7). This might suggest that temporary Erk phosphorylation occurs as a normal process after dissection/surgery and is not affected by CA4P in this context. This experiment was repeated in BIE cells, which do not experience any potential stress associated with dissection before CA4P exposure. In this instance, phospho-Erk levels were elevated with CA4P treatment immediately after the end of CA4P exposure, but had returned to control levels at 1 h after the end of CA4P exposure. Therefore, it could be hypothesized that, in primary cells, a general stress response due to dissection will mask CA4P's effect on Erk phosphorylation. Phosphorylated p38 and JNK levels were not affected by CA4P treatment (data not shown).

We claim:

1. A method of treating or preventing posterior capsule opacification comprising administering a therapeutically effective amount of a combretastatin to a subject suffering from or at risk of developing posterior capsule opacification.

2. The method of claim 1, wherein the combretastatin is combretastatin A-4, or a derivative, prodrug or analog thereof.

3. The method of claim 1, wherein the combretastatin is combretastatin A-4 phosphate (CA4P) or a pharmaceutically acceptable salt thereof.

4. The method of claim 1, wherein the combretastatin is administered systemically.

5. The method of claim 1, wherein the combretastatin is administered non-systemically.

6. The method of claim 5, wherein the combretastatin is administered topically to the eye of the subject.

7. The method of claim 6, wherein the combretastatin is administered during surgery to insert an intraocular lens.

8. Use of a combretastatin for the manufacture of a medicament for the treatment or prevention of posterior capsule opacification.

9. The use of claim 8, wherein the combretastatin is combretastatin A-4, or a derivative, prodrug or analog thereof.

10. The use of claim 8, wherein the combretastatin is combretastatin A-4 phosphate (CA4P) or a pharmaceutically acceptable salt thereof.

11. The use of claim 8, wherein the medicament is suitable for systemic administration.

12. The use of claim 8, wherein the medicament is suitable for topical administration to the eye of a subject suffering from or at risk of developing posterior capsule opacification.
